(19) United States
(12) Patent Application Publication
(10) Pub. No.: US 2010/0004694 A1
(43) Pub. Date: Jan. 7, 2010

(54) Screw assembly

(75) Inventor: Thomas Carter Little, Encinitas, CA (US)

Correspondence Address: ALPHATEC SPINE, INC., 5818 EL CAMINO REAL, CARLSBAD, CA, 92008, US

(73) Assignee: ALPHATEC SPINE, INC., Carlsbad, CA (US)

(21) Appl. No.: 12/459,521

(22) Filed: Jul. 1, 2009

Related U.S. Application Data
(60) Provisional application No. 61/133,731, filed on Jul. 1, 2008.

(51) Int. Cl. A61B 17/86

(52) U.S. Cl. 606/308; 606/309

(57) ABSTRACT

Apparatus and method of using a screw assembly. The screw assembly includes a pedicle screw having a head portion and a shaft portion adapted to be secured to a vertebrae, and a body having pivotally coupled side portions configured to pivot from a first configuration for receiving the head portion of the screw into the body to a second configuration for engaging the head portion of the screw in a locking arrangement with the body.

### CROSS-REFERENCE TO RELATED APPLICATIONS

The present application claims priority to U.S. Provisional Patent Application No. 61/133,731 to Little, filed Jul. 1, 2008, and entitled “A SCREW ASSEMBLY”, the disclosure of which is incorporated herein by reference in its entirety. The present application relates to the U.S. Pat. No. 7,377,923 to Purcell et al., filed May 19, 2004, issued May 27, 2008, and entitled “Variable Angle Spinal Screw Assembly”, and incorporates its disclosures herein by reference in its entirety.

### BACKGROUND OF THE INVENTION

1. Field of the Invention

The present invention generally relates to the field of spinal surgery. In particular, the present invention relates to the field of surgical access to the spine. More particularly, the present invention relates to an apparatus for internal fixation of the spine and a novel locking mechanism for a spinal screw assembly.

2. Background

Certain spinal conditions, including a fracture of a vertebra and a herniated disc, indicate treatment by spinal immobilization. Several methods of spinal joint immobilization are known, including surgical fusion and the attachment of pins and bone plates to the affected vertebras. One known device is a bone interface anchor inserted into at least two spaced-apart vertebras, with a stabilization rod interconnecting the two or more anchors to stabilize the vertebras spanned by the anchors. Specifically, a bone screw is received within a socket formed in the anchor. The anchor further includes a channel, extending perpendicular to the longitudinal axis of the bone screw, for receiving the stabilization rod. The anchor further comprises a threaded portion above the channel. After the bone screw and anchor have been inserted into the bone material, the rod is placed within the channel and a nut is mated with the external threads of the anchor. The nut applies a compressive force between the rod and the screw head to firmly fix the rod between the spanned vertebras and thus stabilize the spinal vertebrae.

During surgical implantation of these prior art stabilization systems, the surgical site is crowded with tissue masses, sponges and other surgical implements that obstruct access to the anchor threads. Given the difficult access, it is possible for the surgeon to cross-thread the nut with the threads of the anchor after the fixation rod is in place. If the threads of the anchor are cross-threaded, the cross-threaded coupling must be removed and replaced before the surgery can proceed. In addition, the threaded fastener (e.g., the nut) is frequently removed and then reinstalled as the surgeon makes progressive bends to contour the fixation rod. This increases the surgery with each on-off iteration and further increases the chances of cross-threading.

Another problem associated with threaded attachments is the torque exerted on the anchor during the tightening of the threaded fastener about the upper end portion of the fixation device. This torque can inadvertently introduce stress points along the rod, bend the rod or even loosen the threaded engagement of the anchor in the bone. The elimination of the conventional threaded attachments in the fixation device of the present invention also obviates these problems associated with torquing.

The angle at which the anchor screws extend from the vertebra pedicle is dictated by the spinal curvature, the orientation of individual vertebra within the spine, and the surgeon's placement of the screw within the pedicle. For example, there is considerable spinal curvature in the region of the S1 L5 vertebra junction and the angle between the longitudinal axis of the screws and the vertebra in that region vary over a wide range. Also, it may be necessary to displace one or more of the anchors from the spin midline to effectuate maximum spinal stabilization. Thus, the rod-receiving channels are typically not collinear nor coplanar and, the rod must be shaped or contoured by the surgeon during the implantation procedure to fit within the channels along the spinal column. The prior art systems allow the coupling unit to pivot with respect to the screw over a range of about ±20° to ±30°, providing some margin for the surgeon to place the rod within the channel.

One challenge with current variable angle or polyaxial systems is aligning the coupling units in a manner that minimizes pre-insertion rod contouring while allowing the surgeon maximum range to optimize pedicle screw placement. This is especially challenging when fusing the S1-L5 junction. The prior art coupling units allow only a limited range of motion with respect to the screw head. The present invention allows a first range of motion in all directions, but also provides an extended range of motion in the medial-lateral-inferior direction (head-to-toe). This extended range of motion, as compared to the prior art, allows the surgeon additional freedom in locating the screws and eases the assembly process by reducing the requirement for rod contouring.

Thus, the present invention provides an extended range of motion as compared to the prior art, allowing the surgeon additional freedom in locating the screws and easing the assembly process by reducing the requirements for rod contouring. The present invention additionally eliminates the numerous problems heretofore experienced with threaded fasteners. The result is a significantly improved variable angle spinal screw assembly.

### SUMMARY OF THE INVENTION

In some embodiments, the present invention relates to a screw assembly. The screw assembly includes a pedicle screw having a head portion and a shaft portion adapted to be secured to a vertebrae and a body having pivotally coupled side portions configured to pivot from a first configuration for receiving the head portion of the screw into the body to a second configuration for engaging the head portion of the screw in a locking arrangement with the body.

In some embodiments, the pedicle screw head portion is substantially spherical.

In some embodiments, the side portions include rounded interior surfaces configured to engage the screw head in a ball joint arrangement. The ball joint arrangement allows variable angular movement of the body with respect to the pedicle screw.

In some embodiments, the screw assembly further includes a fixation rod, wherein in the second configuration the body further comprises a body cavity between the side portions configured to accept the fixation rod.

In some embodiments, the fixation rod within the body cavity prohibits the side portions of the body from pivoting to the first configuration.

In some embodiments, the screw assembly further includes a locking cap releasably securable within the body cavity and configured to lock the fixation rod to the body member. The locking cap includes serrations configured engage serrations on the side portions within the body cavity.

In some embodiments, the screw assembly further includes a spring configured to bias the side portions toward the second configuration

In some embodiments, the screw assembly further includes a locking clip configured to engage the side portions and lock the body in the second configuration

In some embodiments, each side portion includes a pin and a pin receiving hole that engage one another at the pivot when the side portions are assembled

In some embodiments, the present invention relates to a screw assembly. The assembly includes a screw and a body member having a first portion and a second portion disposed between a bottom portion and a top portion of the body member, the first portion and the second portion are pivotally coupled to each other. Upon pivoting the first and second portions in a first direction, the body member is configured to receive the screw at the bottom portion of the body member and upon receipt of the screw, the first and second portions are configured to be pivoted in a second direction, thereby clamping the screw at the bottom portion of the body member.

In some embodiments, the present invention relates to a method of using a screw assembly secured to a vertebra for use with a fixation rod. The method includes a screw assembly comprising a pedicle screw having a head portion and a shaft portion adapted to be secured to a vertebrae and a body having pivotally coupled side portions configured to pivot from a first configuration for receiving the head portion of the screw into a screw-loading portion of the body to a second configuration for engaging the head portion of the screw in a locking arrangement with the body, inserting the pedicle screw into the vertebrae, pivoting the coupled side portions to the first configuration, placing the screw head into the screw-loading portion of the body, pivoting the coupled side portions to the second configuration engaging the head portion of the screw, inserting the fixation rod into a body cavity between the side portions configured to accept the fixation rod in the second configuration, and inserting a locking cap into the body cavity, the locking cap being configured to lock the fixation rod to the body member.

In some embodiments, engaging the head portion of the screw includes a ball joint arrangement allowing variable angular movement of the body with respect to the pedicle screw.

### BRIEF DESCRIPTION OF THE DRAWINGS

The present invention is described with reference to the accompanying drawings. In the drawings, like reference numbers indicate identical or functionally similar elements.

FIG. 1 is a perspective view of an exemplary variable angle spinal screw assembly, according to some embodiments of the present invention.

FIG. 2 is a perspective view of an exemplary bone screw portion of the assembly, according to some embodiments of the present invention.

FIGS. 3A and 3B are perspective views of an exemplary body member of the assembly, according to some embodiments of the present invention.

FIG. 4 is another perspective view of the body member showing the lower surface thereof.

FIG. 5 is a perspective view of an exemplary bushing employed in the assembly, according to some embodiments present invention.

FIGS. 6A and 6B are perspective views of an exemplary cap, according to some embodiments of the present invention.

FIG. 7 illustrates another exemplary cap, according to some embodiments of the present invention.

FIG. 8 is a side view of an exemplary variable angle spinal screw assembly, according to some embodiments of the present invention.

FIG. 9A is a sectional view taken along the line A-A of FIG. 8

FIG. 9B is a sectional view taken along the line B-B of FIG. 8.

FIG. 10 is an exploded view of an exemplary modified form of the pedicle screw and body member, according to some embodiments of the present invention.

FIG. 11 is a perspective view of the modified pedicle screw and body member of FIG. 10 shown in the attached position prior to threading the body member over the screw head to form the mating relationship between the spherical lower portion of the screw head and the interior lower surface of the body member.

FIG. 12 is an exploded perspective view of another modified form of the pedicle screw and body member, according to some embodiments of the present invention.

FIG. 13 is a representational side view of the embodiment of the pedicle screw and body member shown in FIG. 12 with the body member on the screw in the mating variable angle position.

FIG. 14 illustrates an exemplary embodiment of a bottom loading screw and rod assembly in an open configuration, according to some embodiments of the present invention.

FIG. 15 illustrates an exemplary embodiment of the bottom loading screw and rod assembly shown in FIG. 14 in a closed configuration, according to some embodiments of the present invention.

FIGS. 16A-16D are perspective views of assembly of one embodiment of the screw assembly of the present invention.

FIGS. 17A and 17B are perspective views of the pivoting body member of the assembly of the present invention.

### DETAILED DESCRIPTION OF THE INVENTION

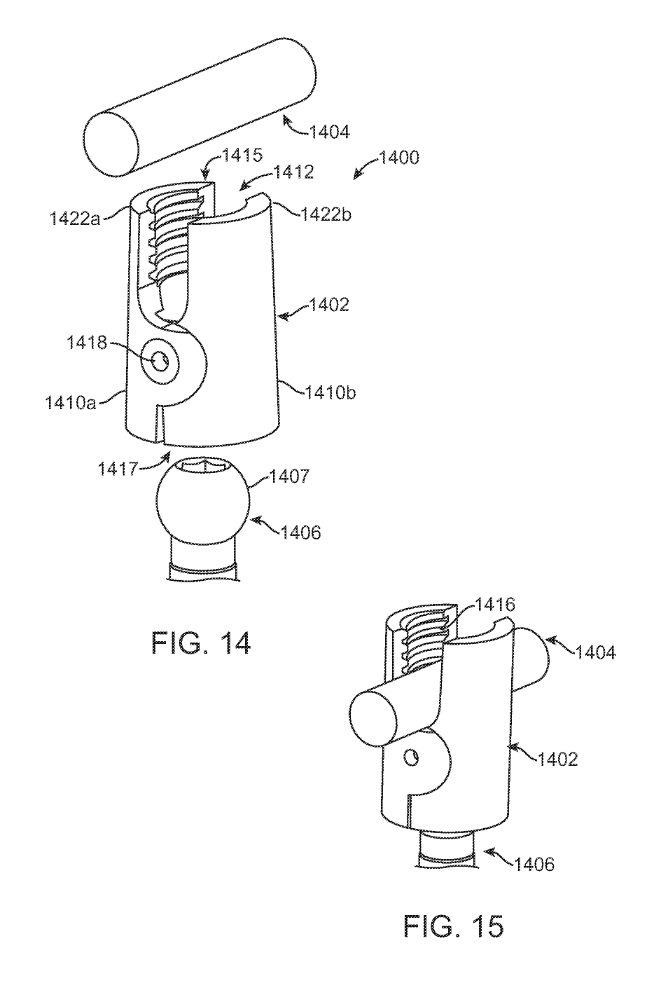

The present invention relates to a bottom loading screw and rod assembly shown in FIGS. 14-16D. Referring to FIG. 14, a screw-and-rod assembly 1400 is shown, according to some embodiments of the present invention. The assembly 1400 includes a body 1402, a rod 1404, and a screw 1406. The body 1404 includes a top rod-loading portion 1415 and a bottom screw-loading portion 1417. The rod 1404 is configured to be secured at the top of the body 1402 by loading the rod 1404 into the top rod-loading portion 1415. The screw 1406 is configured to be loaded at the bottom through the bottom-loading portion 1417. The body 1402 includes two side portions 1410a and 1410b pivotally secured to each other using a pivot 1418. The pivot 1418 allows top surfaces 1422a, 1422b of the top portion 1415 to approach one another, as shown in FIG. 14, this movement allows the bottom portion 1417 to open to a first configuration for receiving the head portion of the screw 1406. The top surfaces 1422a, 1422b move also away from each other, as shown in FIG. 15, to a second configuration for engaging the head portion of the screw 1406 in a locking arraignment. By moving side portions 1410a and 1410b away from one another, the rod 1404 is configured to be inserted into a body cavity 1412, thereby securing the screw 1406 in the body 1402.

The pedicle screw 1406 may include a spherical head 1407 having defining a slot, recess or other means that may be used to drive the screw into vertebrae or bone. The round spherical head 1407 mates with rounded interior surfaces 1430 formed in the lower end of side portions 1410a and 1410b, when assembled, to form a modified ball joint that provides the desired variable angular movement of the body 1402 with respect to the embedded pedicle screw 1406. The threaded shaft portion 1407 of screw extends therefrom through the bottom portion 1417 in the lower end of body 1402. The assembled system provides an extended range of motion of the body 1402 with respect to the pedicle screw 1406. In one embodiment, the range of motion is about ±30 degree in all directions (as measured from the longitudinal axis of the screw) and about ±40 degree in the inferior-superior direction, the outwardly (as viewed from the screw head) concave surfaces provide the ±40 degree range of motion, for a total motion range of 80 degree. This extended range of motion, as compared to the prior art, allows the surgeon additional freedom in locating the screws and eases the assembly process by reducing the requirement for a rod contouring.

As shown in the figures, when side portions 1410a, 1410b of body 1402 are assembled in the second configuration, a pair of channels or opposed parallel slots are formed in body cavity 1412. The slots are sized to receive the fixation rod therein as shown in the drawings. Thus, during assembly, the surgeon may exert a slight downward force on the rod, snapping the rod into the transverse channel defined by the aligned slots.

The interior surface of side portions 1410a, 1410b the body 1402 may also have radially projecting serrations formed therein defining a plurality of axially aligned ratchet teeth or threads 1416. To secure the fixation rod 1404 within the body 1402, a locking cap 1424 is provided. The cap 1424 includes a plurality of radially projecting serrations, ratcheting teeth or threads to engage the ratchet teeth or threads 1416 of side portions 1410a and 1410b. In one embodiment, the cap thread engages the thread of the side portions 1410a and 1410b and is screwed down until a bottom surface of the cap abuts the fixation rod 1404 and presses the rod into cavity 1412. In another embodiment, upon pressing the cap 1424 downwardly into body member 1402, the ratcheting teeth allow the cap to be pressed downwardly but not retracted. As cap is pressed downwardly into the body member, a bottom surface of the cap abuts the fixation rod 1404 and presses the rod into cavity 1412. The interlocked ratchet teeth will allow the surgeon to tighten the clamping force on the fixation rod by simply pressing downwardly on the locking cap. The teeth will hold the component parts in place. Some embodiments allow adjustment of the rod. To adjust or remove the rod, the locking cap is simply rotated 90 degrees about its longitudinal axis, whereupon teeth on the cap are aligned with the open slots in the body member, allowing the cap to be simply pulled upwardly away from the fixation rod. A hexagonally configured slot may be provided in the top portion of cap to facilitate the rotation of the locking cap with a suitably sized mating tool.

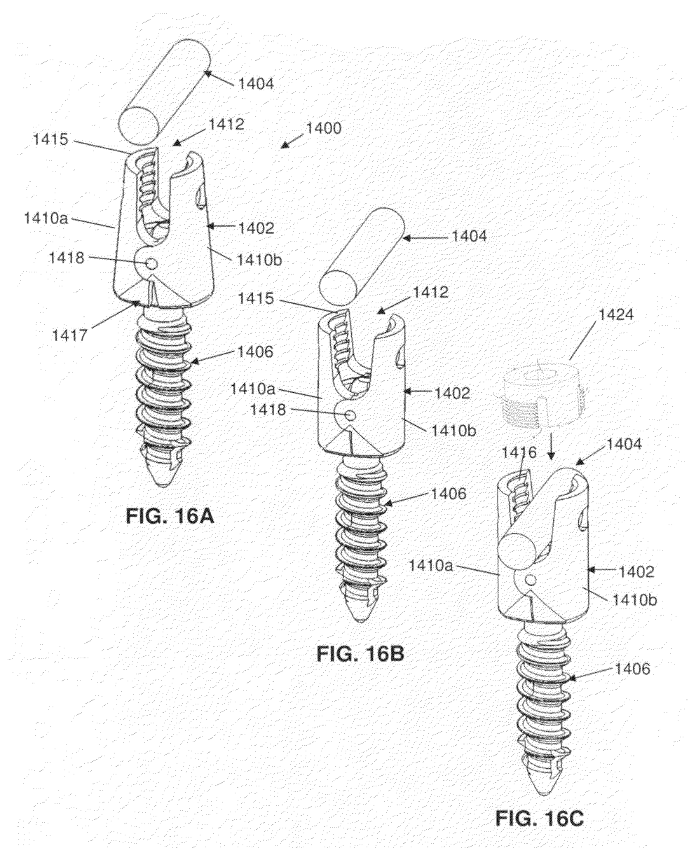

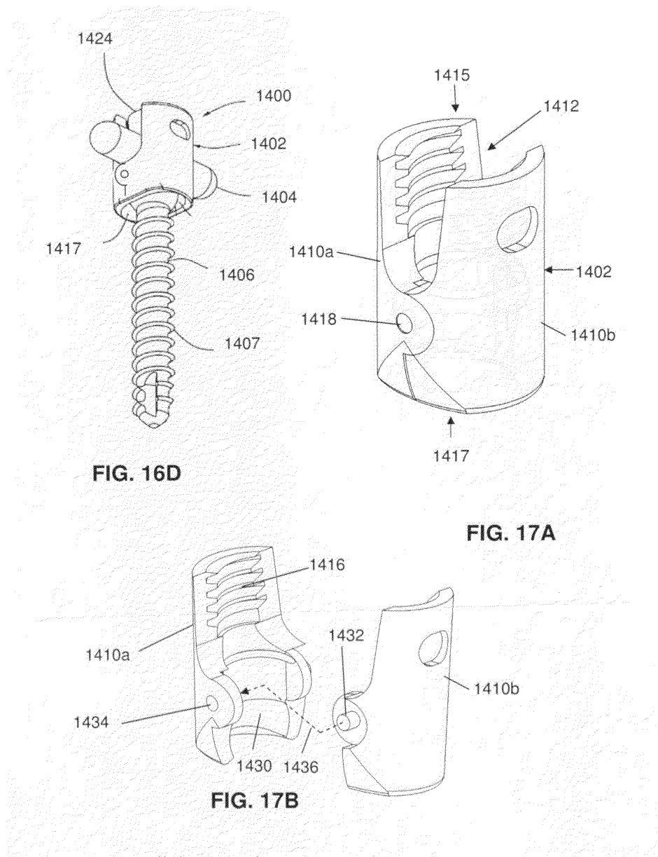

FIGS. 16A-16D show loading of the screw 1406 into the body 1402. A surgeon (or any other medical professional) would push together portions 1410a, 1420b at their top portions 1422 toward each other, opening the bottom portion 1417 into the first configuration, then the screw 1406 may be loaded into the bottom portion 1417, shown in FIG. 16A. Once the screw 1406 is loaded, the surgeon can force the portions 1410 apart (spread out) into a second configuration, shown in FIG. 16B. The rod 1404 is then inserted into the body cavity 1412, shown in FIG. 16C, thereby creating a locking arrangement between the body 1402 and the screw 1406. A cap 1424 is then inserted into the opening 1412, securing the rod 1404 in place, shown in FIG. 16D. In some embodiments, the opposed slots formed between side portions 1410a, 1410b may be tapered such that the tighter the rod is inserted into the body cavity 1412, the greater the frictions on the screw head.

FIGS. 14-17B illustrate two portions 1410a, 1410b for the body 1402 for exemplary purposes only. As can be understood by one skilled in the art, there can be a plurality of portions (e.g., four), thereby creating a multi-portion locking arrangement that locks the screw at the bottom portion of the body 1402 and allowing insertion of rods or any other devices at the top portion of the body 1402. In the embodiment of multi-portion arrangement, the body 1402 includes a plurality of pivots that allow pivoting of each individual portion.

FIGS. 17A and 17B show more detail of the body 1402. The side portions 1410a and 1410b of the body 1402 are designed to engage one another at a pivot 1418, with each portion having a pin 1432 and a pin receiving hole 1434. To assemble, the two portions 1410a and 1410b are brought close together and each pin is aligned with each hole and inserted, such as along line 1436. This allows both portions 1410a and 1410b to be the same. Once assembled, portions 1410a and 1410b are pivotally coupled to each at pivot 1418. The body 1404 also includes a top rod-loading portion 1415 and a bottom screw-loading portion 1417. A spring may be coupled to the portions 1410a and 1410b to keep the body biased in the first or second configuration. In other words, the body 1402 has a clothespin type design, where squeezing the top surfaces 1422a, 1422b toward each other opens the bottom screw-loading portion 1417 to fit over the head portion of the screw. When the top surfaces 1422a, 1422b are released, the spring moves the surfaces away from each other and the bottom screw-loading portion 1417 closes over the head portion of the screw. In some embodiments, a locking clip may also be used to engage the side portions and lock the body in the second configuration once the screw is engaged.

The pivot 1418 allows top surfaces 1422a, 1422b of the top portion 1415 to approach one another, as shown in FIG. 14, this movement allows the bottom portion 1417 to open to the first configuration for receiving the head portion of the screw 1406. The top surfaces 1422a, 1422b may also move away from each other, as shown in FIG. 15, to a second configuration for engaging the head portion of the screw in a locking arraignment. By moving away from one another to the second configuration, the rod 1404 may be inserted into a body cavity 1412, thereby securing the screw 1406 in the body.

In use, at least two of the pedicle screws 1406 are inserted into the vertebra pedicles spanning the vertebra to be fixated. The side portions 1410a, 1410b of body 1402 are pivoted to the first configuration for receiving the screw head, the body 1402 is then lowed over the screw head 1407 and the side portions 1410a, 1410b are pivoted to the second configuration, engaging the head portion of the screw in a locking arraignment with the body. The surgeon preliminary contours the fixation rod 1404 and checks the alignment between the rod 1404 and the channels or opposed parallel slots formed between side portions 1410a, 1410b in the second configuration. Since additional contouring is usually required to improve the alignment, the surgeon incrementally adjusts the rod shape and checks the fit within the channels until the rod properly fits in all channels. During the contouring process, a locking cap 1424 can be mated with one or more of the body 1402 (by pressing the cap 1424 axially into the body member to create the interlock between the ratchet teeth on the body member and the cap) to temporarily hold the rod in place, thereby assisting the surgeon in achieving an accurate fit. The locking caps are then easily removable (by rotating the cap a quarter of a turn to disengage the interlocking teeth), allowing the rod to be further contoured. Once properly contoured, the rod is inserted into the channels and a locking cap is pressed tightly into each body member and bushing to secure the rod in place. To effect securement of the rod at each of the pedicle screw assemblies, it is solely necessary to press the locking cap longitudinally into the body member such that the bottom surface of the cap presses against the fixation rod, causing the rod to press downwardly and against the head of the pedicle screw.

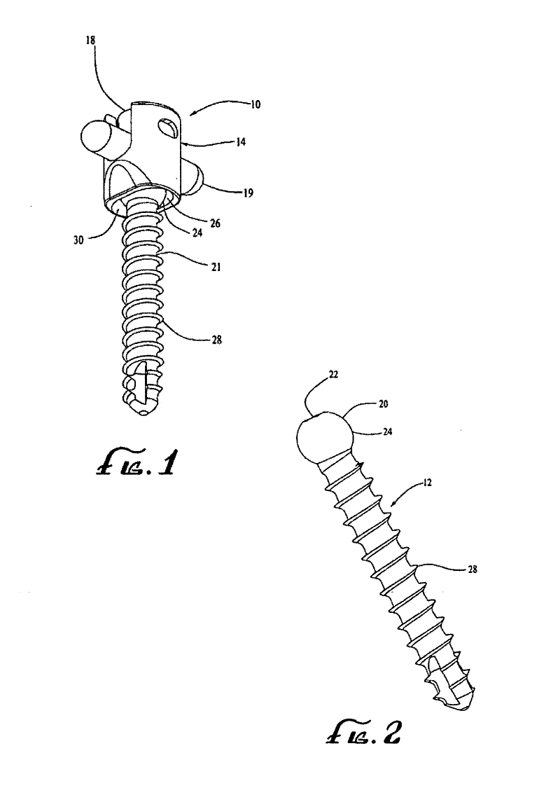

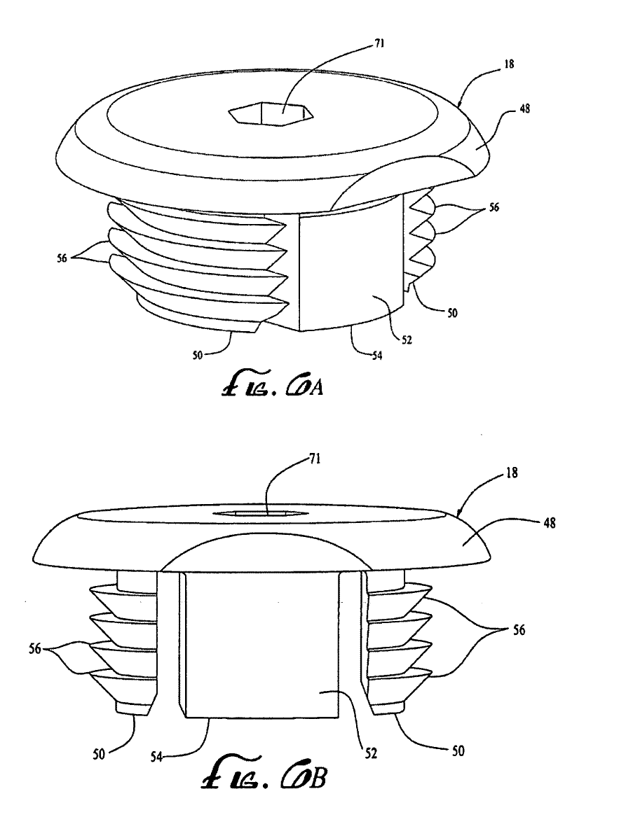

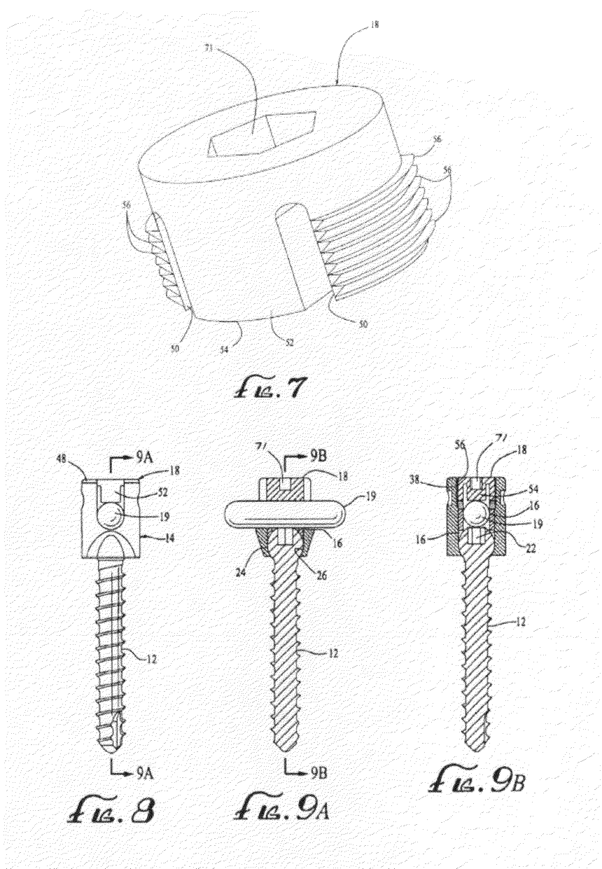

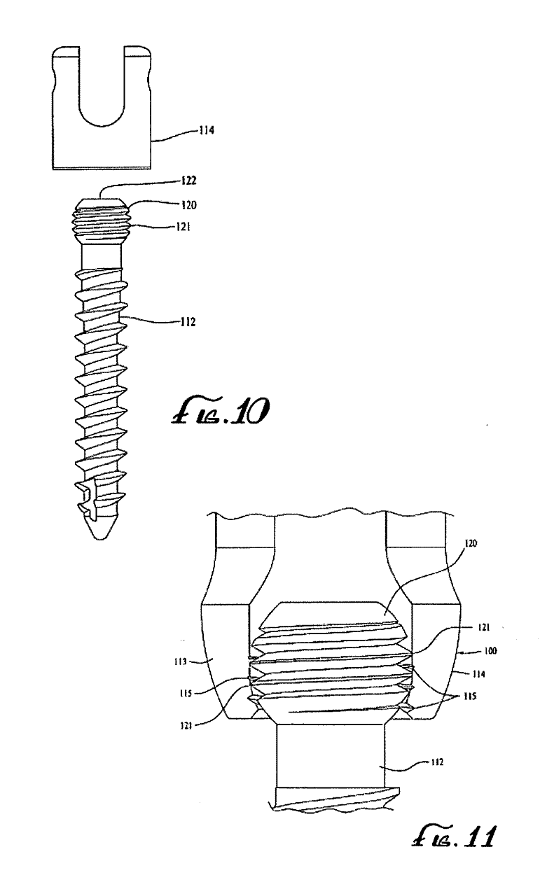

In some embodiments, the body 1402 includes a tapered top portion that allows the body 1402 to open up (splay or spread out) at the bottom portion 1417 to allow loading of the screw 1406 (with the head of the screw 1406 being inserted into the bottom portion 1417 of the body) prior to clamping the rod 1404 in place. In some embodiment, the cavity 1412 can include threading 1416 disposed near the top portion of the body 1402. An exemplary cavity disposed inside the body 1402 for rod-loading and screw loading is disclosed in co-owned U.S. Pat. No. 7,377,923 to Purcell et al., the disclosure of which is incorporated hereby reference and reiterated below. This patent also discloses loading of variable angle spinal screws. As can be understood by one skilled in the art, other types of screws can be loaded into the screw assembly shown in FIGS. 14-16D, for example, those shown in FIGS. 10-13. In other embodiments, the screw assembly may be used without the rod and/or cap and attach to other medical devices or equipment requiring secure attachment of a screw and body to the spine. The assemblies illustrated in FIGS. 1-13 are provided here for exemplary non-limiting purposes only.

Referring now in detail to the drawings, the variable angle spinal screw assembly 10 of the present invention comprises a pedicle screw 12, a body member 14, a bushing 16 and a locking cap 18. The assembly 10 is used with at least one other such assembly and a stabilization or fixation rod 19 to connect the assemblies and stabilize the vertebras into which the assemblies are inserted. The pedicle screw 12 preferably employed in assembly 10 has a spherical head 20 defining a slot 22 therein used to drive the screw into the bone. The rounded surface 24 defined by the lower portion of screw head 20 rests upon and mates with a rounded interior surface 26 formed in the inner or lower end of the body member 14 of the assembly 10 so as to form a modified ball joint that provides the desired variable angular movement of the body member with respect to the embedded pedicle screw. The threaded shaft portion 28 of screw 12 extends therefrom through the opening 30 in the lower end of body member 14.

The body member 14 of assembly 10 further defines a pair of opposed parallel slots 32 axially disposed in the side wall 34 thereof, which terminate at their lower ends in curvilinear surfaces 36. The two slots 32 are sized to receive the fixation rod therein as shown in the drawings with the walls 35 defining the slots preferably extending upwardly beyond the midpoint of the rod and can be inclined slightly to provide a slight holding force on the rod prior to securing the rod with the locking cap 18. Thus, during assembly, the surgeon exerts a slight downward force on the rod, snapping the rod into the transverse channel defined by the aligned slots 32.

The outer or upper interior surface of side walls 34 of the body member 14 both have radially projecting serrations formed therein defining a plurality of axially aligned ratchet teeth 38. The exterior bottom surface 40 of body member 14 has spaced outwardly extending concave surface 42 formed therein and a pair of perpendicularly disposed concave surfaces 44. Surfaces 42 and 44, together with mating surfaces 24 and 26 on the screw head and body member of the assembly, provide an extended range of motion of the body member 14 with respect to the pedicle screw 12. In one embodiment, the range of motion is about ±30 degree in all directions (as measured from the longitudinal axis of the screw) and about ±40 degree in the inferior-superior direction, the outwardly (as viewed from the screw head) concave surfaces provide the ±40 degree range of motion, for a total motion range of 80 degree. This extended range of motion, as compared to the prior art, allows the surgeon additional freedom in locating the screws and eases the assembly process by reducing the requirement for a rod contouring.

To secure the fixation rod 19 within the body member 14 of the assembly, locking cap 18 is provided. Cap 18 defines a top portion 48, a pair of opposed arcuate depending leg portions 50 and a centrally disposed depending projection 52 equidistantly spaced from leg portions 50. Central projection 52 preferably defines a planar lower or bottom surface 54. The leg portions 50 of cap 18 each have a plurality of radially projecting serrations formed therein that define a plurality of axially aligned ratchet teeth 56 adopted to engage teeth 38 on the opposed interior side walls 34 of the body member 14, as will be described.

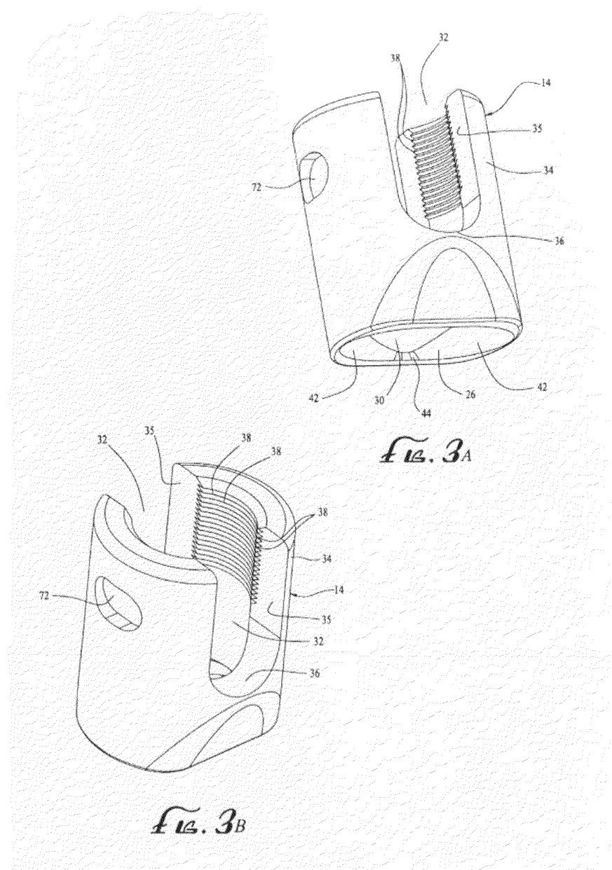

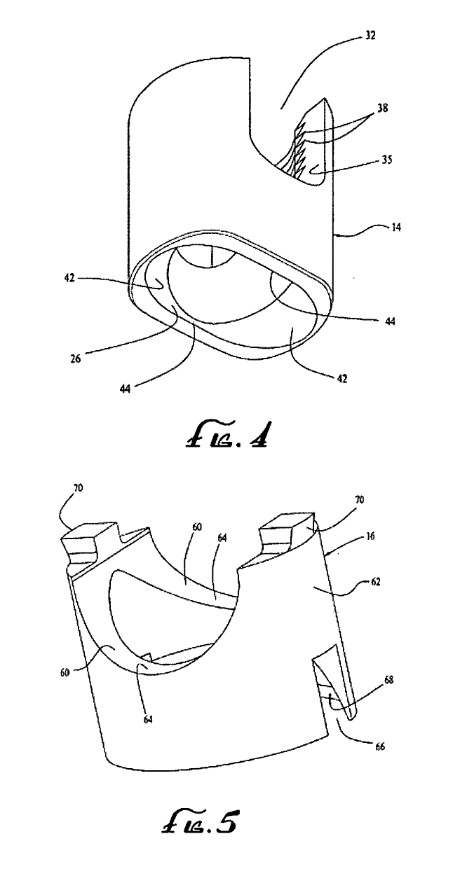

A bushing 16 is preferably employed within the body member 14 of the assembly 10 adjacent side walls 34 to better distribute the longitudinal forces exerted on the pedicle screw. Bushing 16 defines a pair of opposed concave surfaces 60 formed in the upper end of a circular skirt 62 so as to define a seat 64 for the fixation rod 19. The lower portion of bushing skirt 62 is slotted at 66 to provide flexibility therein and defines depending tapered end surfaces 68 adapted to abut opposed sides of the rounded screw head 20. A pair of outwardly projecting opposed resilient tabs 70 are provided at the upper ends of the bushing 16 between concave surfaces 60 that are adapted to be received in a snap fitment within a pair of opposed apertures 72 formed in the side wall 34 of body member 14 whereupon the rod seat 64 in bushing 16 is aligned with the channel in the body member. Note that only one of apertures 72 is illustrated in FIGS. 3A and 3B to better illustrate the configuration of the ratchet teeth 38. In an alternative embodiment, the tabs could be removed from the bushing 16 (as seen in FIG. 4) and located on the body member 14 for engagement with apertures or other receiving members formed in opposed sides of the bushing.

To provide a basic stability to the system during initial assembly, the bushing 16 with its slotted lower skirt portion can be configured to provide a press fitment about the screw head 20 so that the pedicle screw 12, body member 14 and bushing 16 will not move freely prior to the insertion and securement of the fixation rod. In addition, the upper portion of the bushing could be configured such that the wall surfaces 60 defining the rod seat 64 therein extend upwardly past the midpoint of the rod and are slightly inwardly inclined. This would provide the same slight holding force when the rod is pushed into the bushing seat 64 that was above described with reference to the channel walls 35 in the body member 14 of the assembly 10.

Upon securing the bushing 16 in the body member 14 and the fixation rod 12 in bushing seat 64, the locking cap 18 is positioned such that the depending leg portions 50 thereon are aligned with the side walls 34 of body member 14. Upon pressing the cap 18 downwardly into body member 14, the ratchet teeth 38 and 56 on the assembly body and cap interlock so as to allow the cap to be pressed downwardly but not retracted. As cap 18 is pressed downwardly into the body member of the assembly, the planar bottom surface 54 of the central projection 52 thereon abuts the fixation rod 19 and presses the rod into and against the seat 64 formed on the upper end of bushing 16. The resulting pressure on the bushing causes the tapered surfaces 68 on the lower end of the bushing to press against the rounded surface of the screw head 20, thereby securing the rod in seat 64 and providing a decentralized and evenly distributed force acting along the longitudinal axis of the screw. Thus, the use of bushing 16 creates a taper lock between the pedicle screw and body member and increases the area of contact therebetween. The result is an improved locking securement over that provided by the earlier described direct contact of the fixation rod against the upper end of the screw head.

The interlocked ratchet teeth will allow the surgeon to tighten the clamping force on the fixation rod by simply pressing downwardly on the locking cap 18. The teeth will hold the component parts in place. To adjust or remove the rod 19, the locking cap 18 is simply rotated 90 degrees about its longitudinal axis, whereupon the teeth 38 on the depending leg portions 50 of the cap are aligned with the open slots 32 in the body member 14, allowing the cap to be simply pulled upwardly away from the fixation rod 19. A hexagonally configured slot 71 is provided in the top portion 48 of cap 18 to facilitate the rotation of the locking cap with a suitably sized mating tool.

In use, at least two of the pedicle screws 12 with the body members 14 and attached bushings 16 disposed about the screw are inserted into the vertebra pedicles spanning the vertebra to be fixated. The surgeon preliminary contours the fixation rod and checks the alignment between the rod and the mating channels formed by the slots in the bushing and body member of the assemblies. Since additional contouring is usually required to improve the alignment, the surgeon incrementally adjusts the rod shape and checks the fit within the channels until the rod properly fits in all channels. During the contouring process, a locking cap 18 can be mated with one or more of the body member 14 (by pressing the cap axially into the body member to create the interlock between the ratchet teeth on the body member and the cap) to temporarily hold the rod in place, thereby assisting the surgeon in achieving an accurate fit. The locking caps are then easily removable (by rotating the cap a quarter of a turn to disengage the interlocking teeth), allowing the rod to be further contoured. Once properly contoured, the rod is inserted into the channels and a locking cap is pressed tightly into each body member and bushing to secure the rod in place. To effect securement of the rod at each of the pedicle screw assemblies, it is solely necessary to press the locking cap longitudinally into the body member such that the bottom surface 54 of the central projection 52 on the cap presses against the fixation rod 19, causing the rod to press downwardly against the bushing 16, which in turn mates with and presses against the head of the pedicle screw.

A modified form of the variable angle spinal screw assembly is illustrated in FIGS. 10 and 11. This modified form of the assembly enables the surgeon to insert the pedicle screw in the bone, by itself, unencumbered by the body member. In the prior embodiment, the pedicle screw 12 must be inserted through the body member 14 before the screw can be driven into the bone. With the body member attached, securement of the screw into the bone can be somewhat difficult. In the modified assembly 100, the outer surface of the spherical head portion 120 of the pedicle screw 112 is provided with threads 121, as seen in FIG. 10. As in the prior embodiment, the upper end of head portion 120 is provided with a vertical slot 122 used to drive the screw into place. The lower interior portion 113 of the body member 114 to be used with the modified pedicle screw 112 is provided with threads 115 adapted to engage threads 121 on the screw. As a result, the body member 114 can be threaded onto (see FIG. 11) and over the head 120 of the screw 112 after the screw is driven into place. With the exception of threads 121 and 115, the pedicle screw 112 and body member 114 are identical in configuration to the screw 12 and body member 14 of the prior embodiment. Thus, after the body member 114 is threaded onto and over the screw head and is disposed within the interior of the lower end of body member 114, as seen in FIG. 11, the variable angular relationship therebetween is formed as in the prior embodiment.

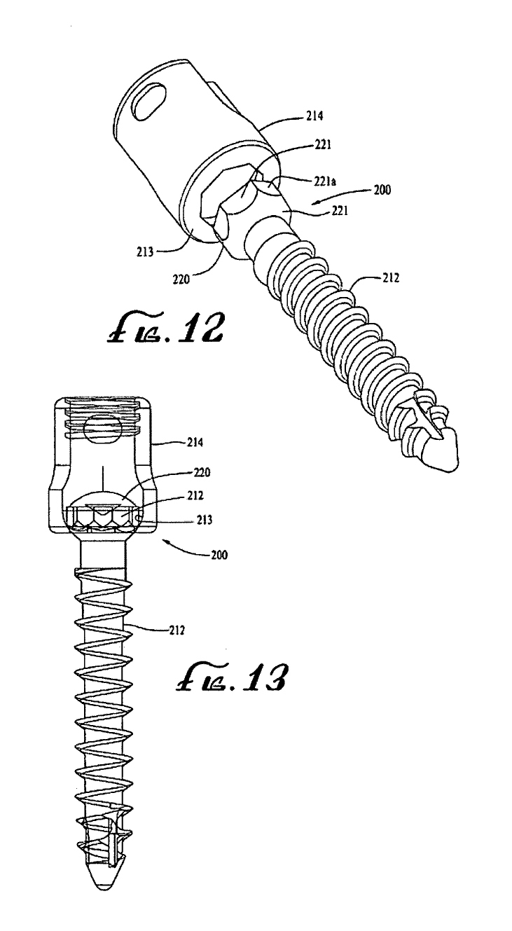

A second modified form of the variable angle spinal screw assembly that enables the surgeon to insert the pedicle screw in the bone, by itself, unencumbered by the body member is illustrated in FIGS. 12 and 13. As seen therein, the mating threads on the pedicle screw 112 and body member 114 have been replaced with mating octagonal surfaces. In this second modified assembly 200, the outer surface of the spherical head portion 220 of the screw 212 is provided with an octagonal portion. The octagonal portion is comprised of eight contact surfaces 221, one of which (e.g. 221a) is unequal in length to the remaining surfaces. The lower interior portion 213 of the body member 214 to be used with the modified pedicle screw 212 is also provided with an octagonally configured portion adapted to engage and mate with the octagonal surfaces on the screw head. Because of the inclusion of a differently-sized surface on both the screw and body member, the pedicle screw 212 will only align with the body member 214 in only one position, i.e., where the shortened contact surface on the screw head is aligned with the correspondingly shortened surface in the lower interior of the body member. Accordingly, the pedicle screw 212 can again be inserted into the bone without being attached to the body member 214. After the screw 212 is driven into place, the body member 214 can be inserted over the screw head with the octagonal surfaces thereon aligned with the corresponding surfaces on the screw head. By pressing the screw body downwardly, it is completely inserted onto the screw head and the mating octagonal surfaces are moved out of engagement. Upon rotating the body member and pulling upwardly on the body member, such that the head is disposed within the interior of the lower end of body member 214 the variable angular relationship therebetween illustrated in FIG. 13 is formed as in the prior embodiments. It is to be understood that this form of the present invention is not limited to the use of mating octagonal surfaces. Any polygonal configuration could be employed on the screw head and body member wherein at least one of the mating surfaces on the screw head and on the body member is correspondingly off-sized or otherwise differently configured from the remaining surfaces on the screw head and body member.

In another embodiment of the invention, the bushing 16 is not employed. The opposed axial slots 32 in the side wall 34 of the body member of the assembly define a seat for the fixation rod 19. When the locking cap is pressed into the body member with the fixation rod extending thereacross, the planar bottom surface 54 of the central projection 52 again abuts the fixation rod and, in this instance, presses the rod against the upper end of the head of the pedicle screw. For such applications, the body member and pedicle screw would be sized such that the upper surface of the screw would project above the bottom of the seat defined by the axially opposed slots 32 so as to enable the rod to press against the screw and create a rigid, yet adjustable, securement between the body member and the pedicle screw. This embodiment can also be utilized with the modified forms of the pedicle screw 128 and body member 114 shown in FIGS. 10 and 11. In all of these embodiments, the components of the variable angle spinal screw assembly are preferably formed of titanium.

It should be noted that while the preferred configuration of the locking cap provides a rounded and flush mounting on the upper ends of the body member 14 when the locking cap is fully inserted against the fixation rod, other locking cap configurations could be employed. For example, FIG. 7 illustrates a locking cap having a generally cylindrical perimeter portion in which the ratchet teeth 56 project radially therefrom along leg portions 50. This configuration is illustrated in FIG. 1. As a result, the upper end of the locking cap would be inwardly offset from the upper end of the body member without adversely effecting the operation of the variable angle spinal screw assembly. Various other changes and modifications also could be made in carrying out the present invention.

Example embodiments of the methods and components of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

What is claimed:

1. A screw assembly, comprising:
a pedicle screw having a head portion and a shaft portion adapted to be secured to a vertebrae; and
a body having pivotally coupled side portions configured to pivot from a first configuration for receiving the head portion of the screw into a bottom screw-loading portion of the body to a second configuration for engaging the head portion of the screw in a locking arrangement with the body.

2. The assembly of claim 1, wherein the pedicle screw head portion is substantially spherical.

3. The assembly of claim 1, wherein the side portions include rounded interior surfaces configured to engage the screw head in a ball joint arrangement.

4. The assembly of claim 3, wherein the ball joint arrangement allows variable angular movement of the body with respect to the pedicle screw.

5. The assembly of claim 1, further comprising a fixation rod, wherein in the second configuration the body further comprises a body cavity between the side portions configured to accept the fixation rod.

6. The assembly of claim 5, wherein the fixation rod within the body cavity prohibits the side portions of the body from pivoting to the first configuration.

7. The assembly of claim 5, further comprising a locking cap releasably securable within the body cavity and configured to lock the fixation rod to the body member.

8. The assembly of claim 7, wherein the locking cap includes serrations configured to engage serrations on the side portions within the body cavity.

9. The assembly of claim 1, further comprising a spring configured to bias the side portions toward the second configuration.

10. The assembly of claim 1, further comprising a locking clip configured to engage the side portions and lock the body in the second configuration.

11. The assembly of claim 1, wherein the each side portion includes a pin and a pin receiving hole that engage one another at the pivot when the side portions are assembled.

12. A screw assembly, comprising:
a screw;
a body member having a first portion and a second portion disposed between a bottom portion and a top portion of said body member, said first portion and said second portion are pivotally coupled to each other;
wherein upon pivoting said first and second portions in a first direction, said body member is configured to receive said screw at said bottom portion of said body member and upon receipt of said screw, said first and second portions are configured to be pivoted in a second direction, thereby clamping said screw at said bottom portion of said body member.

13. The assembly of claim 12, wherein a fixation device is configured to be inserted into said top portion of said body member further securing said head portion inside said body member.

14. The assembly of claim 12, wherein said screw includes a head portion configured to be inserted into and clamped by said bottom portion of said body member.

15. The assembly of claim 12, wherein said body portion includes a plurality of pivotally arranged portions configured to clamp said screw at said bottom portion of said body member.

16. A method of using a screw assembly secured to a vertebra for use with a fixation rod, the method comprising:
providing a screw assembly comprising:
a pedicle screw having a head portion and a shaft portion adapted to be secured to a vertebrae; and
a body having pivotally coupled side portions configured to pivot from a first configuration for receiving the head portion of the screw into a screw-loading portion of the body to a second configuration for engaging the head portion of the screw in a locking arrangement with the body;
inserting the pedicle screw into the vertebrae;
pivoting the coupled side portions to the first configuration, placing the screw head into the screw-loading portion of the body;
pivoting the coupled side portions to the second configuration engaging the head portion of the screw;
inserting the fixation rod into a body cavity between the side portions configured to accept the fixation rod in the second configuration; and
inserting a locking cap into the body cavity, the locking cap being configured to lock the fixation rod to the body member.

17. The method of claim 16, wherein engaging the head portion of the screw includes a ball joint arrangement allowing variable angular movement of the body with respect to the pedicle screw.
